(19) United States
(12) Patent Application Publication
(10) Pub. No.: US 2020/0063106 A1
(43) Pub. Date: Feb. 27, 2020

(54) CELL CULTURE MEDIUM, AND CELL CULTURE APPARATUS AND CELL CULTURE METHOD EACH USING SAME

(71) Applicant: HITACHI, LTD., Tokyo (JP)

(72) Inventors: Keisuke SHIBUYA, Tokyo (JP); Masashi MARUYAMA, Tokyo (JP); Takeyuki KONDOU, Tokyo (JP)

(21) Appl. No.: 16/462,954

(22) PCT Filed: Oct. 31, 2017
(86) PCT No.: PCT/JP2017/039337

§ 371 Date: May 22, 2019

(30) Foreign Application Priority Data
Nov. 25, 2016 (JP) 2016-228559

(51) Int. Cl. C12N 5/071; C12M 1/06; C12M 1/00; C12M 1/34

(52) U.S. Cl. CPC C12N 5/0682; C12M 27/02; C12N 2533/30; C12M 41/12; C12N 2500/30; C12M 29/00

(57) ABSTRACT

A cell culture apparatus (1) of the present invention comprises: a culture vessel (2) in which a cell culture medium comprising at least one of culture components composed of a conjugate with a stimuli-responsive polymer is stored and cells are cultured in the medium; a stimulus-applying mechanism (4) that applies a predetermined stimulus to the conjugate so as to induce a predetermined change of the stimuli-responsive polymer in response to the stimulus; and, a separation mechanism (5) that separates at least a part of the medium components except for the conjugate from the cell culture medium, while leaving the conjugate in the cell culture medium, on the basis of a property change of the stimuli-responsive polymer.

### TECHNICAL FIELD

The present invention relates to a cell culture medium, and a cell culture apparatus and a cell culture method each using the same.

### BACKGROUND ART

There are a variety of cell culture methods widely known, designed to culture cells including animal cells, plant cells and microbial cells in predetermined culture media to produce and collect useful substances. Among these methods, continuous culture is advantageous over batch culture and fed-batch culture, since a collection process for collecting useful substances may be allowed to proceed continuously, in parallel with a culture process. Such continuous culture can improve productivity of the useful substances by presetting the cell density per unit volume of medium at higher levels.

Among prior methods of continuous culture, one known method is such that cells drawn out from a culture vessel together with an useful substance is returned back to the culture vessel, in order to keep the cell density in the culture vessel at high levels (see Patent Literature 1, for example).

### CITATION LIST

### Patent Literature

Patent Literature 1: JP-A-2016-054686

### SUMMARY OF INVENTION

### Technical Problem

The prior continuous culture (see Patent Literature 1, for example) needs, however, additional feeding of a fresh liquid medium to the culture vessel, corresponding to the volume of liquid medium having been drawn out. The prior continuous culture has thus suffered from a problem that production cost of the useful substance to be produced would be high.

Therefore, an object of the present invention is to provide a cell culture medium which may reduce the production cost of a useful substance obtained by cell culture compared to before, and a cell culture apparatus and a cell culture method each using the same.

### Solution to Problem

A cell culture medium of the present invention, successfully solved the aforementioned problem, comprises at least one of medium components that is composed of a conjugate with a stimuli-responsive polymer.

Moreover, a cell culture apparatus of the present invention has successfully solved the aforementioned problem and comprises: a culture vessel that stores a cell culture medium and culture cells, wherein at least one culture component of the cell culture medium is composed of a conjugate with a stimuli-responsive polymer; a stimulus-applying mechanism that applies a predetermined stimulus to the conjugate so as to induce a predetermined change of the stimuli-responsive polymer in response to the stimulus; and, a separation mechanism that separates at least a part of the medium components except for the conjugate from the cell culture medium, while leaving the conjugate in the cell culture medium, on the basis of a property change of the stimuli-responsive polymer.

Moreover, a cell culture method of the present invention has successfully solved the aforementioned problem and comprises: a cell culture step in which cells are cultured in a cell culture medium having at least one of the medium components composed of a conjugate with a stimuli-responsive polymer; a stimulus-applying step in which a predetermined stimulus is applied to the conjugate so as to induce a predetermined change of the stimuli-responsive polymer; and, a separating step in which at least a part of the medium components except for the conjugate is separated from the cell culture medium, while leaving the conjugate in the cell culture medium, on the basis of a property change of the stimuli-responsive polymer.

### Advantageous Effects of Invention

The present invention has successfully provided a cell culture medium which may reduce the production cost of a useful substance obtained by cell culture compared to before, and a cell culture apparatus and a cell culture method each using the same.

### BRIEF DESCRIPTION OF DRAWINGS

FIG. 1 is an explanatory structural drawing illustrating a cell culture apparatus according to an embodiment of the present invention.

FIG. 2 is a schematic drawing of a conjugate that composes the cell culture medium according to an embodiment of the present invention, showing a conjugate of a stimuli-responsive polymer with a cell growth factor.

FIG. 3 is an explanatory structural drawing illustrating a specific example of the conjugate.

FIG. 4A is a schematic drawing illustrating a state of the conjugate illustrated in FIG. 2, in a cell culture step included in the cell culture method according to an embodiment of the present invention. FIG. 4B is a schematic drawing illustrating a state of the conjugate illustrated in FIG. 2, in a stimuli-applying step included in the cell culture method according to an embodiment of the present invention.

FIG. 5 is a schematic drawing of a conjugate that composes the cell culture medium according to another embodiment of the present invention, showing a conjugate of a stimuli-responsive polymer, with a binding factor that binds a cell growth factor.

FIG. 6 is a schematic drawing illustrating a state of the conjugate illustrated in FIG. 5, in the stimuli-applying step included in the cell culture method according to another embodiment of the present invention.

FIG. 7 is a structural explanatory drawing illustrating a specific example of a conjugate after being stimulated.

FIG. 8 is a structural explanatory drawing illustrating a cell culture apparatus used in Examples of the present invention.

FIG. 9 is a illustration of a intracellular mechanism of action of a first conjugate prepared in Example.

FIG. 10 is a illustration of a intracellular mechanism of action of a second conjugate prepared in Example.

### DESCRIPTION OF EMBODIMENTS

Embodiments of the present invention will be explained below, properly referring to the drawings. The embodiments are, however, not limited to the details below, and may be conducted in a suitably modified manner within the scope of the present invention.

The cell culture medium of the present invention is mainly featured by that the culture medium is composed of a conjugate with a stimuli-responsive polymer. Moreover, the cell culture apparatus and the cell culture method are mainly featured by that they uses such cell culture medium.

First, an overall structure of the cell culture apparatus according to an embodiment of the present invention will be explained, which will be followed by explanations of the cell culture medium used for the cell culture apparatus, and the cell culture method.

### «Cell Culture Apparatus»

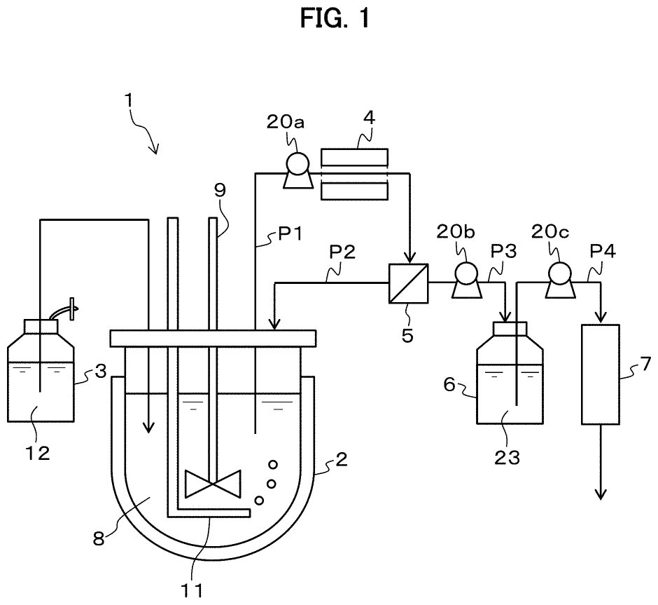

FIG. 1 is a structural explanatory drawing illustrating a cell culture apparatus 1 according to this embodiment.

As illustrated in FIG. 1, the cell culture apparatus 1 mainly has a culture vessel 2, a supplementary medium vessel 3, a stimulus-applying mechanism 4, a separation mechanism 5, a reservoir 6, and a purification mechanism 7.

### <Culture Vessel>

The culture vessel 2 is a component that receives and keeps a supplementary medium 12, which is a liquid medium (culture fluid) fed from the supplementary medium vessel 3, and cultures cell in a cell culture medium 8. Materials for composing the culture vessel 2 are exemplified by, but not limited to, metals such as stainless steel and aluminum, synthetic resins such as polypropylene and polystyrene, and glass. The cell culture medium 8 will be detailed later.

The culture vessel 2 is equipped with a stirrer 9 that stirs a content of the culture vessel 2. The stirrer 9 stirs the content housed in the culture vessel 2 into uniformity.

The culture vessel 2 is also equipped with an aeration unit 11, such as sparger, which blows a gas such as oxygen, nitrogen or carbon dioxide into the content of the culture vessel 2. Another aeration unit is disposed also in an upper space in the culture vessel 2, although not illustrated. Conditions to be set for aeration through these units into the culture vessel 2 include type of gas depending on environments for cell culture, and for a case where two or more types of gas are used, ratio and flow rates of these gases.

Although not illustrated, the culture vessel 2 is also equipped with a temperature control mechanism that keeps the cell culture medium 8 at a predetermined temperature (suitable temperature for culturing). The culture vessel 2 may still also be equipped with instruments for measuring, for example, dissolved oxygen concentration, dissolved carbon dioxide gas concentration, pH, temperature and so forth, and instruments for measuring concentration of cell metabolites (waste products) such as ammonia, lactic acid and glutamic acid.

Note that the culture vessel 2 in this embodiment is kept under a gauge pressure of 0.01 to 0.05 MPa or around, in order to prevent, for example bacterial invasion, from the outside.

### <Supplementary Medium Vessel>

The supplementary medium vessel 3 is connected to the culture vessel 2, and feeds the supplementary medium 12, which is a liquid medium (culture fluid), to the culture vessel 2 as described above. The supplementary medium 12 may have a composition similar to that of filtrate of the cell culture medium 8 obtained through the later-described separation mechanism 5, after drawn out from the culture vessel 2 together with the useful substance, but excluding unnecessary substances such as waste products, typically as described later.

Note that the volume of the supplementary medium 12 fed from the supplementary medium vessel 3 to the culture vessel 2 in this embodiment may be set equal to the volume of the aforementioned filtrate separated through the separation mechanism 5.

Materials for composing the supplementary medium vessel 3 are exemplified by, but not limited to, metals such as stainless steel and aluminum, synthetic resins such as polypropylene and polystyrene, and glass.

### <Stimulus-Applying Mechanism>

The stimulus-applying mechanism 4 is provided close to a pipe P1 through which the cell culture medium 8 stored in the culture vessel 2 is pumped out to the separation mechanism 5. Note that, in FIG. 1, reference symbol 20a denotes a feed pump provided to the pipe P1.

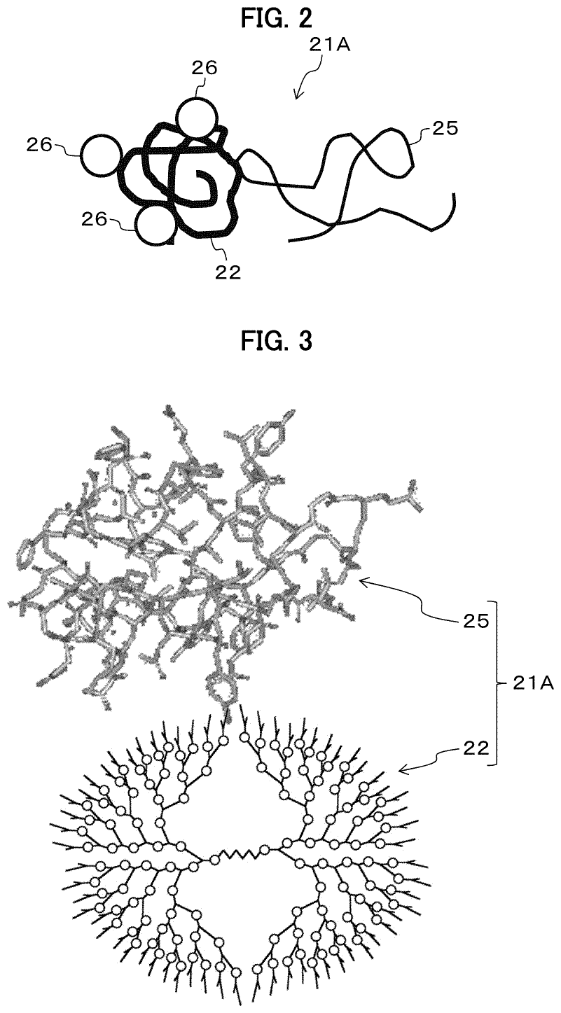

The stimulus-applying mechanism 4 is designed to apply a predetermined stimulus to the cell culture medium 8 that flows through the pipe P1. More specifically, stimulus is applied to a stimuli-responsive polymer 22 (see FIG. 2) of a later-described conjugate 21A (see FIG. 2) contained in the cell culture medium 8.

The stimuli-responsive polymer 22 changes its structure, potential, hydrophilicity/lipophilicity in response to the stimulus applied by the stimulus-applying mechanism 4.

The stimulus is selected depending on types of the stimuli-responsive polymer 22 (see FIG. 2) described later, such as temperature responsive polymer, pH responsive polymer, ionic strength responsive polymer, photoresponsive polymer, magnetic field responsive polymer, electric field responsive polymer, and dynamic stimulation responsive polymer. That is, the stimulus is exemplified by a temperature change, a pH change, an ionic strength change, a light intensity change, a strength change in a magnetic field and an electric field, and a dynamic stimulation change.

For the cell culture apparatus 1 of this embodiment, the temperature responsive polymer is assumed as the stimuli-responsive polymer 22 (see FIG. 2). The stimulus-applying mechanism 4 in this embodiment is therefore constructed using a temperature regulation mechanism (for example, Peltier element, heater, etc.) that regulates temperature of the cell culture medium 8 flowing through the pipe P1 to a predetermined value (at around 37° C.).

Note that the stimulus-applying mechanism 4 used in the cell culture apparatus 1 of the present invention is not limited to the temperature regulating mechanism, instead allowing for example a pH regulating mechanism, anionic strength regulating mechanism, a light intensity regulating mechanism, a magnetic field strength regulating mechanism, an electric field strength regulating mechanism, or a dynamic stimulation strength regulating mechanism to be used, depending on types of the stimuli-responsive polymer 22.

### <Separation Mechanism>

The separation mechanism 5 separates, by filtration, cells (cultured cells) and a predetermined medium component (later-described conjugate 21A (see FIG. 2)) from a cell culture medium 8 (liquid medium) that was drawn out together with a useful substance from the culture vessel 2, leaving the residual components of the cell culture medium 8 in a filtrate.

As the separation mechanism 5 of this embodiment, a filter, which has a pore size which does not allow cells (cultured cells) and the conjugate 21A to pass there through but allow the residual components of the cell culture medium 8 other than the aforementioned matters to pass there through, may be used.

As this sort of separation mechanism 5, preferable is a structure using an ultrafiltration membrane. A hollow fiber membrane module is particularly preferable.

The cells (cultured cells) and the conjugate 21A separated by the separation mechanism 5 are returned through the pipe P2 back to the culture vessel 2.

The other residual components of the cell culture medium 8 separated in the filtrate by the separation mechanism 5 is pumped through a pipe P3 to the reservoir 6. Now in FIG. 1, reference symbol 20b denotes a feed pump provided to the pipe P3.

The residual components of the cell culture medium 8 include the useful substance produced by cell culture in the culture vessel 2, cell metabolites (waste products), and medium components other than the conjugate 21A.

The useful substance is exemplified by, but not limited to, proteins such as antibodies and enzymes; and physiologically active substances. If the useful substance is intracellularly produced, such useful substance is separated and extracted from the cell that is collected in some other process.

<Reservoir>

The reservoir 6 is composed of a container that temporarily stores a filtrate 23 separated by the separation mechanism 5. Materials for composing the reservoir 6 are exemplified by, but not limited to, metals such as stainless steel and aluminum, synthetic resins such as polypropylene and polystyrene, and glass.

Note that the reservoir in this embodiment is kept under a gauge pressure of 0.01 to 0.05 MPa or around using an unillustrated pressure regulating valve, pump or the like, in order to prevent for example bacterial invasion from the outside.

<Purification Mechanism>

The purification mechanism 7 of this embodiment is connected via a pipe P4 to the reservoir 6. In FIG. 1, reference symbol 20c denotes a feed pump provided to the pipe P4.

The purification mechanism 7 purifies a useful substance contained in the filtrate 23 pumped out from the reservoir 6.

The purification mechanism 7 is exemplified by, but not limited to, an affinity chromatograph, a high performance liquid chromatograph, an ion exchange chromatograph, and an gel filtration chromatograph. Two or more types of purification mechanism 7 may be used in a combined manner.

The useful substance purified by the purification mechanism 7 is eluted by a properly selected eluent, and collected in an unillustrated collection container. Meanwhile, buffer solutions used for washing and equilibrating the purification mechanism 7 are stored in an unillustrated waste container, and discarded after proper waste treatment.

### «Cell Culture Medium»

Next, the cell culture medium 8 of this embodiment (see FIG. 1) will be explained.

The cell culture medium 8 is a liquid medium as described above, and is composed of an aqueous solution or aqueous dispersion that contains various components (medium components) that make up a growth environment necessary for a predetermined cell when cultured therein.

As will be detailed later, in this embodiment, at least one of the medium components contained in the cell culture medium 8 is composed of a conjugate 21A (see FIG. 2) with a stimuli-responsive polymer 22 (see FIG. 2).

By the way, the medium components are exemplified by, but not limited to, carbon sources such as molasses, glucose, fructose, maltose, sucrose, starch, lactose, glycerol and acetic acid; nitrogen sources such as corn steep liquor, peptone, yeast extracts, meat extracts, ammonium salt and amino acids; phosphate sources such as monosodium phosphate, disodium phosphate, monopotassium phosphate and dipotassium phosphate; inorganic salts such as sodium chloride, magnesium chloride, magnesium sulfate, ferrous sulfate, ferric sulfate, ferrous chloride, ferric chloride, iron citrate, iron ammonium sulfate, calcium chloride, calcium sulfate, zinc sulfate, zinc chloride, copper sulfate, copper chloride, manganese sulfate and manganese chloride; sulfur sources; bases or nucleic acids such as ATP and FAD; and cell growth factors.

Such medium components are properly selected and used, depending for example on types of cell to be cultured, and types of useful substance to be produced by the cultured cells.

The cell in this embodiment are not specifically limited, and are exemplified by biological cells such as animal cells, plant cells, microbial cells and algae cells.

As previously described, at least one of components of the cell culture medium 8 in this embodiment is composed of the conjugate 21A (see FIG. 2) with the stimuli-responsive polymer 22 (see FIG. 2) described later. In particular, the conjugate 21A composed of the stimuli-responsive polymer 22 and a cell growth factor which is a relatively expensive among the aforementioned medium components is preferable since such conjugate can notably demonstrate the effect of the present invention which will be detailed later.

The paragraphs below will explain a case where at least one of the medium components that compose the cell culture medium 8 is a cell growth factor 25 (see FIG. 2), and the cell growth factor 25 and the stimuli-responsive polymer 22 (see FIG. 2) form the conjugate 21A (see FIG. 2).

### <Conjugate>

FIG. 2 is a schematic drawing of the conjugate 21A that composes the cell culture medium 8 (see FIG. 1) according to an embodiment of the present invention.

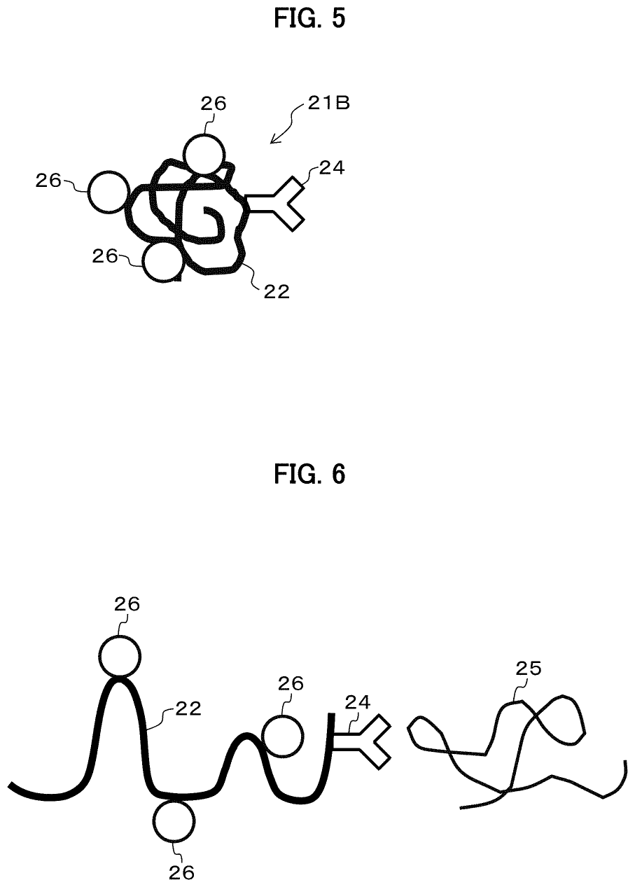

As illustrated in FIG. 2, the conjugate 21A in this embodiment is composed of the stimuli-responsive polymer 22 and the cell growth factor 25 (medium component). Alternatively, the conjugate 21A may be composed of the stimuli-responsive polymer 22 and a binding factor 24 (see FIG. 5), as explained later in other embodiment.

Now in FIG. 2, reference symbol 26 denotes a hydrophilic group introduced into the stimuli-responsive polymer 22. The hydrophilic group will be explained later, following explanations on the stimuli-responsive polymer 22 and the cell growth factor 25.

### (Stimuli-Responsive Polymer)

The stimuli-responsive polymer 22 is a polymer that changes its property such as structure, potential (electric charge) and hydrophilicity/hydrophobicity (physical properties, chemical properties, electric properties, etc.) in response to a predetermined stimulus.

The stimulus is exemplified by, but not limited to, a temperature change, a pH change, an ionic strength change, a light intensity change, an strength change in magnetic, a strength change in electric field, and a dynamic stimulation change.

That is, specific examples of the stimuli-responsive polymer 22 include a temperature responsive polymer, a pH responsive polymer, an ionic strength responsive polymer, a photoresponsive polymer, a magnetic field responsive polymer, an electric field responsive polymer, and a dynamic stimulation responsive polymer, as described previously.

The temperature responsive polymer is preferably any of those having a sharp phase transition temperature or temperature limit at which polymer changes from being distinctively hydrophilic to being distinctively hydrophobic, or vice versa.

The temperature responsive polymer in solution causes changes in conformation or physicochemical properties at so-called critical solution temperature (CST).

LCST-type temperature responsive polymer having lower critical soluble temperature (LCST), when heated from below LCST where the polymer remains hydrophilic, causes decay of the steric structure upon transit of LCST, and turns into hydrophobic.

UCST-type temperature responsive polymer having upper critical soluble temperature (UCST), when heated from below UCST where the polymer remains hydrophobic, turns into hydrophilic upon transit of UCST.

As the stimuli-responsive polymer 22 in the present invention, employable are for example both of the UCST-type temperature responsive polymer such as sulfobetaine polymer which is a bipolar polymer, and the LCST-type temperature responsive polymer exemplified below. The LCST-type temperature responsive polymer is preferable for its easy handleability

The LCST-type temperature responsive polymer is exemplified by, but not limited to, polypropylene glycol, ε-polylysine valeramide, ε-polylysine butyramide, N-hydroxypentyl-ε-polylysine, N-hydroxybutyl-ε-polylysine, polyethylene glycol, poly-N-isopropylacrylamide, poly-N-n-propylacrylamide, poly-N-n-propylmethacrylamide, poly-N,N-diethylacrylamide, poly-N-ethoxyethylacrylamide, poly-N-tetrahydrofurfurylacrylamide, poly-N-tetrahydrofurfurylmethacrylamide, polyvinylcaprolactam, polyvinyl methyl ether, and polymethacrylic ester. One of these LCST-type temperature responsive polymers may be used independently, or two or more of these polymers may be used in a combined manner.

The LCST-type temperature responsive polymer is exemplified by polyamino acid. The polyamino acid forms a helix structure given by a linearly polymerized amino acid chain with the aid of hydrogen bonds.

The polyamino acid is specifically exemplified by, but not limited to, those composed of polypeptide whose polymerization units are amino acids such as glutamic acid, aspartic acid, asparagine, lysine, glutamine, cysteine, alanine, leucine and arginine. The polyamino acid may be either homopolymer or copolymer. One of these polyamino acids may be used independently, or two or more polyamino acids may be used in a combined manner. Among them, at least one of polylysine, polyglutamine and polyarginine is preferable. Dendritic polylysine is particularly preferable.

The number-average molecular weight of the temperature responsive polymer, as the stimuli-responsive polymer 22 (see FIG. 2) in this embodiment, is not specially limited, but is 100 kDa or smaller, so as to be smaller than the cell growth factor 25 (see FIG. 2), and is preferably 5 kDa or smaller. By using the stimuli-responsive polymer 22 with the number-average molecular weight controlled within such ranges, the cell growth factor 25 will fully act and function on the cell.

The temperature responsive polymer in this embodiment also will show a distinctive heat-induced change of response, that is, a structural change between before and after being stimulated. Hence efficiency of separation of the conjugate 21A (see FIG. 2) by the separation mechanism 5 (see FIG. 1) will be improved.

As the temperature responsive polymer in this embodiment, the aforementioned polyamino acid is particularly preferable. When temperature stimulation is applied, the hydrogen bonds in the helix structure of the polyamino acid is cleavage and elongate the amino acid chain. This intensifies the structural change between before and after the stimulation. Since the polyamino acid can relatively easily control orientation of the stimuli-responsive polymer 22 (see FIG. 2), the cell growth factor 25 (see FIG. 2) may easily introduce to a position where allow the cell growth factor to easily bind to a receptor of a cell.

While the cell culture apparatus 1 (see FIG. 1) in this embodiment is assumed to use the temperature responsive polymer as the stimuli-responsive polymer 22 as described above, the present invention alternatively allows any other stimuli-responsive polymer 22 to be used.

Such other stimuli-responsive polymer 22 is exemplified by pH responsive polymer, electric field responsive polymer, photoresponsive polymer, ionic strength responsive polymer, magnetic field responsive polymer, and dynamic stimulation responsive polymer.

The pH responsive polymer is exemplified by, but not limited to, poly (meth) acrylic acid and salt thereof; copolymer of (meth)acrylic acid with (meth)acrylamide, hydroxyethyl (meth)acrylate, alkyl (meth)acrylate or the like, and salts thereof; copolymer of maleic acid with (meth) acrylamide, alkyl (meth)acrylate or the like, and salts thereof; polyvinyl sulfonic acid, polystyrene sulfonic acid and polyvinylbenzene sulfonic acid, and salts thereof; polyacrylamide alkyl sulfonate and salt thereof; polydimethylaminopropyl (meth)acrylamide and salt thereof. One of these pH responsive polymers may be used independently, or two or more of these polymers may be used in a combined manner.

The electric field responsive polymer is exemplified by, but not limited to, poly(amino-substituted (meth) acrylamide), poly(meth)acrylic acid amino-substituted alkyl ester, polyvinylpyridine, polyvinylcarbazol, and polydimethylaminostyrene. One of these electric field responsive polymers may be used independently, or two or more of these polymers may be used in a combined manner.

The photoresponsive polymer is exemplified by polymers that contain azobenzene derivative, spiropyran derivative or triarylmethane derivative, which are capable of causing isomerization reaction.

The ionic strength responsive polymer is exemplified by acrylamide.

The magnetic field responsive polymer is exemplified by magnetic nanoparticle-containing polymer.

The dynamic stimulation responsive polymer is exemplified by agarose. The aforementioned photoresponsive polymer, ionic strength responsive polymer, magnetic field responsive polymer, and dynamic stimulation responsive polymer are not limited to those exemplified above. Also note that one of each of the photoresponsive polymer, ionic strength responsive polymer, magnetic field responsive polymer, and dynamic stimulation-responsive polymer may be used independently, or two or more of each of these polymers may be used in a combined manner.

### (Cell Growth Factor [Medium Components])

Next, the cell growth factor 25 (see FIG. 2) as a representative of the aforementioned medium components will be explained.

The cell growth factor 25 contained as a medium component in the cell culture medium 8 (see FIG. 1) forms the conjugate 21A (see FIG. 2) with the aforementioned stimuli-responsive polymer 22 (see FIG. 2) in the cell culture medium 8.

When the cell growth factor 25 contacts with a predetermined cell (target cell) in the cell culture medium 8, it specifically adsorbs on a receptor which resides on the cell, and thereby induces intracellular signal transduction.

The cell growth factor 25 is exemplified by, but not limited to, epidermal growth factor (EGF), insulin-like growth factor (IGF), transforming growth factor (TGF), nerve growth factor (NGF), brain-derived neurotrophic factor (BDNF), vesicular endothelial growth factor (VEGF), granulocyte-colony stimulating factor (G-CSF), granulocyte-macrophage-colony stimulating factor (GM-CSF), platelet-derived growth factor (PDGF), erythropoietin (EPO), thrombopoietin (TPO), basic fibroblast growth factor (bFGF or FGF2), hepatocyte growth factor (HGF), transforming growth factor (TGF), bone morphogenic protein (BMP), neurotrophin (BDNF, NGF or NT3: brain-derived neurotrophic factor), fibroblast growth factor (FGF), serum, bovine serum albumin (BSA), cholesterols, insulin, and transferrins.

Although the cell growth factor 25 (see FIG. 2) may directly bind to the stimuli-responsive polymer 22 (see FIG. 2), it preferably binds to the polymer via a linker (not illustrated). The cell growth factor 25 may be positioned freely and it may more easily bind to a receptor of a cell in consequence of the cell growth factor 25 binds to the stimuli-responsive polymer 22 via a linker.

The linker is exemplified by biotin-avidin, biotin-streptavidin, and riboflavin-riboflavin. Among them, biotin-avidin is preferable. Biotin-avidin may bind the cell growth factor 25 (see FIG. 2) and the stimuli-responsive polymer 22 (see FIG. 2) more easily. More specifically, the cell growth factor 25 is biotinized, also the stimuli-responsive polymer 22 is biotinized, and thereby the cell growth factor 25 and the stimuli-responsive polymer 22 may easily bind via avidin.

Specific binding site of the cell growth factor 25 (see FIG. 2) on the stimuli-responsive polymer 22 (see FIG. 2) is not specially limited. However from the viewpoint of allowing the cell growth factor 25 to be exposed more reliably on the outer surface of the conjugate 21A (see FIG. 2), the cell growth factor 25 is preferably bound to the terminal of the polymer that composes the stimuli-responsive polymer 22. More specifically, a preferred binding site for the cell growth factor 25 typically is situated on the terminal of a straight chain if the polymer composing the stimuli-responsive polymer 22 has a straight chain form, meanwhile, it is situated on the end of a mesh if the polymer has a mesh-like form.

The cell growth factor 25 may be exposed on the outer surface of the conjugate 21A more surely in consequence of the cell growth factor 25 binds to such sites. In this way, the cell growth factor 25 may adsorbe a receptor of a cell, in the cell culture medium 8 (see FIG. 1) in a specific and efficient manner.

Another method for allowing the cell growth factor 25 (see FIG. 2) to be exposed on the outer surface may be that the cell growth factor 25 adheres on the stimuli-responsive polymer 22 (see FIG. 2) via the aforementioned linker. The method is, however, not limited thereto.

In addition, since the cell growth factor 25 (see FIG. 2) is arranged so as to cover the stimuli-responsive polymer 22 (see FIG. 2), so that the stimuli-responsive polymer 22 may be prevented from non-specifically adhering onto the surface of cell membrane.

That is, as will be explained below, area of non-specific adsorption to the stimuli-responsive polymer 22 may be reduced.

Most cells are likely to non-specifically adsorb on hydrophobic moiety. Hence if the stimuli-responsive polymer 22 (see FIG. 2) that composes the conjugate 21A (see FIG. 2) has much hydrophobic moiety, a portion of a cell surface except for receptors may likely to non-specifically adsorb to such hydrophobic moiety.

In contrast in this embodiment, since the cell growth factor 25 (see FIG. 2) exposes to the outer surface of the conjugate 21A (see FIG. 2), so that the cell growth factor 25 of the conjugate 21A is likely to specifically adsorb onto receptors of a cell. Moreover, by reducing the area of the hydrophobic moiety of the stimuli-responsive polymer 22 (see FIG. 2) in the conjugate 21A, it becomes possible to reduce non-specific adsorption of the stimuli-responsive polymer 22 onto portions of a cell surface except for receptors.

### (Hydrophilic Group)

Next, the hydrophilic group 26 (see FIG. 2) will be explained.

The stimuli-responsive polymer 22 (see FIG. 2) in this embodiment has introduced therein the hydrophilic groups 26, aiming to reduce non-specific adsorption of the stimuli-responsive polymer 22 onto a cell.

Such hydrophilic group 26 is exemplified by functional groups including hydroxy group, carboxy group and phosphate group.

Methods for introducing the hydrophilic group 26 (see FIG. 2) into the stimuli-responsive polymer 22 (see FIG. 2) are exemplified by, but not limited to, methods for co-polymerizing or graft-polymerizing ethylene glycol or polymer thereof (polyethylene glycol), molecule having a phospholipid structure, or phosphoric acid or polymer thereof (polyphosphoric acid), with the stimuli-responsive polymer 22.

A preferred specific example of the stimuli-responsive polymer 22 (see FIG. 2), with the hydrophilic groups 26 (see FIG. 2) introduced therein, is exemplified by polyamino acid given by chemical formula (1) below.

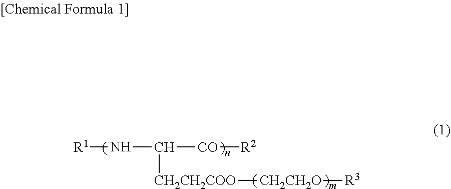

(Where in formula (1), each of R¹, R² and R³ independently represents a hydrogen atom or a lower alkyl group having 1 to 3 carbon atoms, and each of n and m independently represents an integer of 2 or larger.)

Specific site of introduction of the hydrophilic groups 26 (see FIG. 2) on the stimuli-responsive polymer 22 (see FIG. 2) is not specially limited. However from the viewpoint of allowing the hydrophilic groups 26 to expose more reliably onto the outer surface of the stimuli-responsive polymer 22 (see FIG. 2), the hydrophilic groups 26 is preferably situated at the terminal of the polymer that composes the stimuli-responsive polymer 22. More specifically, a preferred site of introduction of the hydrophilic group 26 typically situated at the terminal of a straight chain if the polymer composing the stimuli-responsive polymer 22 has a form of straight chain, meanwhile, it is situated at the end of a mesh if the stimuli-responsive polymer 22 has a mesh-like form.

The hydrophilic groups 26 thus situated into such site may more reliably be exposed on the outer surface of the stimuli-responsive polymer 22. In this way, the hydrophilic groups 26 more reliably reduces non-specific adsorption of the stimuli-responsive polymer 22 onto the cell.

The conjugate 21A (see FIG. 2) is formed by chemical bonding of the cell growth factor 25 (see FIG. 2) to the stimuli-responsive polymer 22 (see FIG. 2).

FIG. 3 is an explanatory structural drawing illustrating a specific example of the conjugate 21A.

As illustrated in FIG. 3, the conjugate 21A is formed by the dendritic polylysine as the stimuli-responsive polymer 22 and insulin as the cell growth factor 25, which are chemically bound to each other.

### «Cell Culture Method»

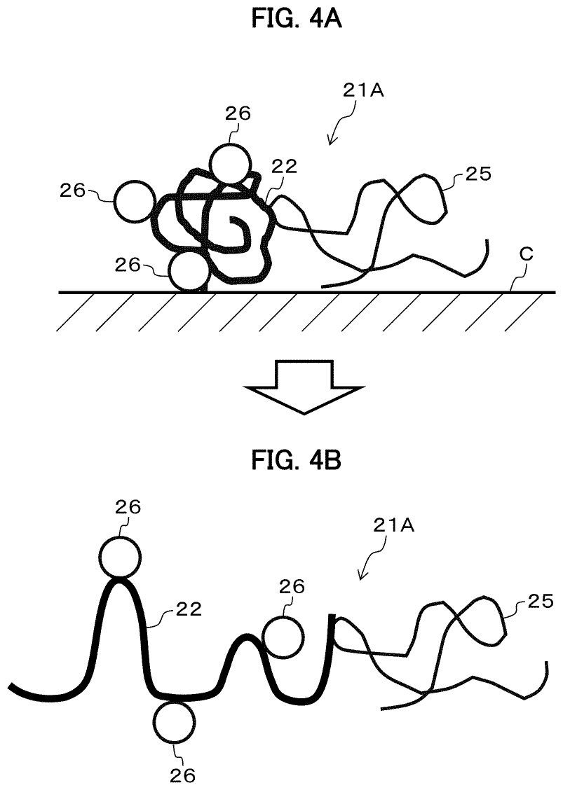

Next, one mode of the cell culture method will be explained mainly referring to FIG. 1 and FIG. 2, without restricting the present invention. FIG. 4A, being occasionally referred to, is a schematic drawing illustrating a state of the conjugate 21A illustrated in FIG. 2, in the culturing step included in the cell culture method. Meanwhile, FIG. 4B is a schematic drawing illustrating a state of the conjugate 21A illustrated in FIG. 2, in the stimuli-applying step included in the cell culture method.

The cell culture method of this embodiment is exemplified by a method that includes (1) culturing step, (2) culture fluid transfer step, (3) stimulus-applying step, (4) separation step, (5) culture fluid returning step, (6) filtrate transfer step, (7) purification step, and (8) medium transfer step.

### (1) Cell Culture Step

First of all, the cell culture in this method is started in the positively pressurized culture vessel 2 (see FIG. 1) that has the cell culture medium 8 (see FIG. 1) composed of an aqueous solution which contains the conjugate 21A (see FIG. 2) and other medium components necessary for the cell culture. Now the conjugate 21A is a product composed of, as described previously, the stimuli-responsive polymer 22 (assuming an LCST-type temperature responsive polymer in this embodiment), and the cell growth factor 25 (see FIG. 2) that corresponds to “at least one of the medium components”, combined as illustrated in FIG. 2.

In the cell culture step, the cell culture is allowed to proceed at a temperature lower than LCST (20° C., for example). Hence the LCST-type temperature responsive polymer that composes the conjugate 21A shows hydrophilicity.

Now, as illustrated in FIG. 4A, the cell growth factor 25 is bound to the stimuli-responsive polymer 22 so as to be exposed on the surface of the conjugate 21A. Moreover, the stimuli-responsive polymer 22 has the hydrophilic groups 26.

With such structure, the cell growth factor 25 of the conjugate 21A can efficiently and specifically bind to the receptor (not illustrated) on a cell C. The cell C can efficiently proliferate in the culture vessel 2 (see FIG. 1).

Inside the culture vessel 2, useful substances and cell metabolites (waste products) are produced in the cell culture medium 8 (see FIG. 1) as the cell C proliferates.

### (2) Culture Fluid Transfer Step

After an elapse of a predetermined number of days after the start of culture, or at a point in time when the concentration of the cultured cell in the cell culture medium 8 (see FIG. 1) reaches a predetermined level, the cell culture medium 8 is pumped out under pressure, from the culture vessel 2 (see FIG. 1) through the pipe P1 (see FIG. 1) to the separation mechanism 5 (see FIG. 1). Pumping under pressure of the cell culture medium 8 is dealt by the feed pump 20a (see FIG. 1) provided to the pipe P1. In this process, a pressurizing device, such as an unillustrated diaphragm pump provided to the separation mechanism 5, may be driven to keep the inside of the separation mechanism 5 at a negative pressure. This enables rapid pumping under pressure of the cell culture medium 8 from the culture vessel 2 to the separation mechanism 5.

### (3) Stimulus-Applying Step

In this step, a predetermined stimulus is applied to the conjugate 21A (see FIG. 2) that is contained in the cell culture medium 8 (see FIG. 1) pumped out from the culture vessel 2 (see FIG. 1) to the separation mechanism 5 (see FIG. 1). In this embodiment, a temperature not lower than LCST (37° C., for example) is applied to the LCST-type temperature responsive polymer as the stimuli-responsive polymer 22 (see FIG. 2) of the conjugate 21A.

As illustrated in FIG. 4B, the stimuli-responsive polymer 22 of the heat-stimulated conjugate 21A causes decay of the steric structure, resulting in alongation of the molecular length and demonstration of hydrophobicity. Now in FIG. 4B, reference symbol 26 denotes a hydrophilic group, and reference symbol 25 denotes a cell growth factor.

By the way, for a case where the aforementioned pH responsive polymer, electric field responsive polymer, photoresponsive polymer, ionic strength responsive polymer, magnetic field responsive polymer, dynamic stimulation responsive polymer or the like is used as the stimuli-responsive polymer 22 (see FIG. 2). The each polymer may change its property such as a steric structure, a potential (electric charge) and hydrophilicity/hydrophobicity by applying stimulation corresponding to each polymer such as a pH change, an electric field strength change, a light intensity change, an ionic strength change, a magnetic field strength change in a magnetic field, and a strength change in a dynamic stimulation.

The cell culture medium 8 (see FIG. 1) that contains the thus stimulated conjugate 21A (see FIG. 2) is pumped to the separation mechanism 5 (see FIG. 1).

### (4) Separation Step

In this step, from the cell culture medium 8 (see FIG. 1) pumped into the separation mechanism 5 (see FIG. 1), the cells and the conjugate 21As (see FIG. 2) with elongated molecular length are separated in the separation mechanism 5 (see FIG. 1). Meanwhile, the residual medium components, useful substances, cell metabolites (waste products), and aqueous medium can pass as a filtrate through the separation mechanism 5. The conjugate 21As now turned into hydrophobic can be filtered off more efficiently, by using a hydrophilic filter membrane. Alternatively, the conjugate 21As now turned to be positively charged upon stimulation in the stimulus-applying step can be filtered off more efficiently, by using a positively charged filter membrane.

### (5) Culture Fluid Returning Step

In this step, the cells and the conjugate 21As (see FIG. 2) having been separated by the separation mechanism 5 (see FIG. 1) are returned back to the culture vessel 2 (see FIG. 1). The cells and the conjugate 21As in this process are returned while being accompanied by a small amount of cell culture medium 8 (see FIG. 1) that remains in the separation mechanism 5. Hence, the cell growth factor 25 (see FIG. 2), which is a relatively expensive medium component, is now recyclable as a result of returning of the conjugate 21As.

The cell culture medium 8 returned back to the culture vessel 2 will contain high concentrations of the cells and the conjugate 21As. Hence, during continuous operation of the cell culture apparatus 1 of this embodiment (see FIG. 1), the concentrations of the cells and conjugate 21As in the culture vessel 2 may be kept at appropriate levels, by periodically repeating the culture fluid returning step.

### (6) Filtrate Transfer Step

The filtrate filtered through the separation mechanism 5 (see FIG. 1) is temporarily stored in the separation mechanism 5. The filtrate typically contains the useful substances and cell metabolites (waste products), as described previously.

In the filtrate transfer step, the filtrate having been stored in the separation mechanism 5 and having reached a predetermined volume is pumped out from the separation mechanism 5, through the pipe P3 (see FIG. 1), into the reservoir 6 (see FIG. 1). For the transfer, employed is a feed pump 20b (see FIG. 1) provided to the pipe P3. In this step, the transfer of the filtrate towards the reservoir 6 may also be accelerated, by keeping the inside of the reservoir 6 at a negative pressure using an unillustrated pump or a pressure regulating valve provided to the reservoir 6.

### (7) Purification Step

In this step, the useful substances, which is contained in the filtrate 23 (see FIG. 1) pumped up from the reservoir 6 (see FIG. 1) to the purification mechanism 7 (see FIG. 1), is purified as described above. The transfer of the filtrate 23 from the reservoir 6 to the purification mechanism 7 is performed when the amount of filtrate 23 stored in the reservoir 6 reached a predetermined volume, with the aid of the feed pump 20c (see FIG. 1) provided to the pipe P4 (see FIG. 1). Alternatively, the transfer of the filtrate 23 towards the purification mechanism 7 may also be accelerated, by keeping the inside of the purification mechanism 7 at a negative pressure using an unillustrated pump or a pressure regulating valve provided on the downstream side of the purification mechanism 7. The purification mechanism 7 can also feed an eluent used for eluting the useful substance from the filtrate 23.

Upon completion of purification of the useful substances, washing buffer, or equilibrating buffer for equilibrating the purification mechanism 7, for example, is fed to the purification mechanism 7, in preparation for the next purification step.

### (8) Medium Transfer Step

In this step, the cell culture medium 8 (see FIG. 1) in the supplementary medium vessel 3 (see FIG. 1) is supplemented to the culture vessel 2 (see FIG. 1).

When the cell culture medium 8 is drawn out together with the useful substances and so forth from the culture vessel 2 towards the separation mechanism 5 (see FIG. 1) as described previously, the level of height of the cell culture medium 8 in the culture vessel 2 descend.

If the level of height becomes lower than a predetermined level, a predetermined volume of the cell culture medium 8 (see FIG. 1) is supplemented from the supplementary medium vessel 3 (see FIG. 1). Note that the amount of supplementation of the cell culture medium 8 from the supplementary medium vessel 3 to the culture vessel 2 (see FIG. 1) is set, as described above, equal to the volume of the filtrate 23 (see FIG. 1) separated by the separation mechanism 5 (see FIG. 1) from the cell culture medium 8 having been drawn out from the culture vessel 2.

Such medium transfer step is carried out concurrently to the individual steps (1) to (7).

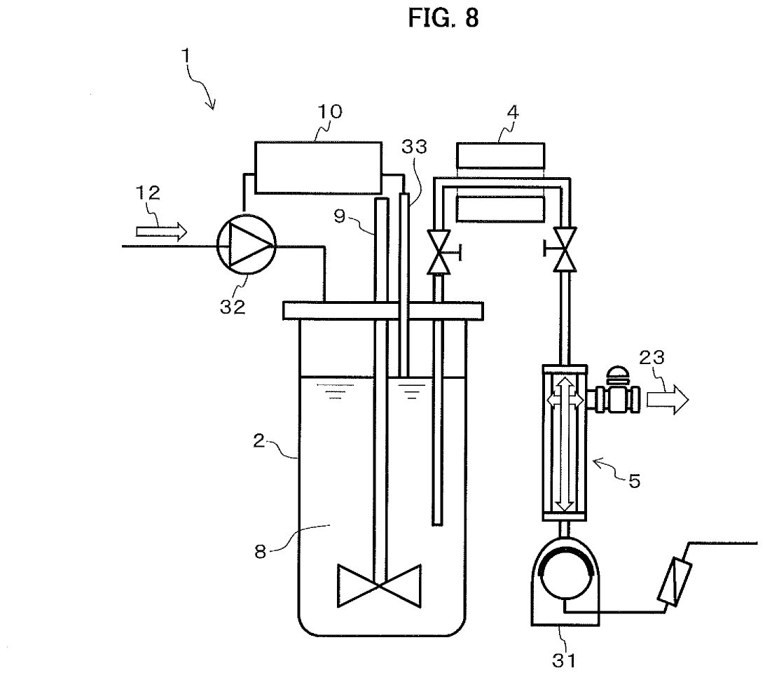

When using the cell culture apparatus 1 (see FIG. 8) described later in Examples, such cell culture method may be carried out while being controlled by a control device 10 (see FIG. 8).

### «Operations and Effects»

Next, operations and effects of this embodiment will be explained.

The cell culture medium 8 (see FIG. 1) of this embodiment contains the medium components, at least one of which is composed of the conjugate with the stimuli-responsive polymer. More specifically, typically as illustrated in FIG. 2, the cell culture medium 8 contains the conjugate 21A composed of the relatively expensive cell growth factor 25 and the stimuli-responsive polymer 22.

By the way, the prior continuous culture (see Patent Literature 1, for example) has needed additional feeding of a fresh liquid medium to the culture vessel, corresponding to the volume of liquid medium having been drawn out from the culture vessel together with the useful substances produced by the cells, as described above. For this reason, the prior continuous culture has suffered from a problem that production cost of producing useful substances would be high.

In contrast, in the cell culture medium 8 (see FIG. 1) of this embodiment, an expensive medium component (cell growth factor 25 (see FIG. 2), for example), binds to the stimuli-responsive polymer 22 (see FIG. 2) which can be separated by the separation mechanism 5 (see FIG. 1) with a predetermined stimulus.

With such cell culture medium 8, the expensive medium component (the cell growth factor 25, for example) may be kept staying in the culture vessel 2 with the aid of the separation mechanism 5, even when the cell culture medium 8 is drawn out together with the useful substances from the culture vessel 2 (see FIG. 1). This successfully reduces the feed volume of the expensive medium component to be supplemented to the culture vessel 2. Hence with the cell culture medium 8 of this embodiment, the production cost of the useful substances obtained by cell culture may be reduced compared to before.

As the stimuli-responsive polymer 22 (see FIG. 2) of the conjugate 21A (see FIG. 2) contained in the cell culture medium 8 (see FIG. 1) of this embodiment, a stimuli-responsive polymer which changes in response to at least one stimulus among from temperature, light and pH may be used.

With such cell culture medium 8, it will become easier to quantitatively control the amount of stimulus applied on the stimuli-responsive polymer 22 (see FIG. 2), and will therefore become easier to control the amount of the changed stimuli-responsive polymer 22 in response to the stimulus. That is, with such cell culture medium 8, the conjugate 21A (see FIG. 2) that contains the expensive medium component (cell growth factor 25 (see FIG. 2), for example) may be separated by the separation mechanism 5 (see FIG. 1) more reliably. As a consequence, the production cost of the useful substances may be reduced than before more reliably.

Alternatively, as the stimuli-responsive polymer 22 (see FIG. 2) of the conjugate 21A (see FIG. 2) contained in the cell culture medium 8 (see FIG. 1) of this embodiment, a stimuli-responsive polymer which, at least, elongates its molecular chain and changes its polarization charge in response to the stimulus.

With such cell culture medium 8, the conjugate 21A (see FIG. 2) that contains the expensive medium component (cell growth factor 25 (see FIG. 2), for example) may be separated by the separation mechanism 5 more reliably. That is, the production cost of the useful substances may be reduced than before more reliably.

Alternatively, as the stimuli-responsive polymer 22 (see FIG. 2) of the conjugate 21A (see FIG. 2) contained in the cell culture medium 8 (see FIG. 1) of this embodiment, employable is at least one of polylysine, polyglutamine, and polyarginine. These stimuli-responsive polymers 22 show relatively large changes in response to between before and after stimulation, and may therefore be separated by the separation mechanism 5 more reliably. That is, the production cost of the useful substances may be reduced than before more reliably.

Moreover, these stimuli-responsive polymers 22 may have the cell growth factor 25 (see FIG. 2) situated at a site that can facilitate specific binding to a receptor of a cell.

Alternatively, as the conjugate 21A (see FIG. 2) contained in the cell culture medium 8 (see FIG. 1) of this embodiment, employable is the conjugate 21A (see FIG. 2) of the stimuli-responsive polymer 22 (see FIG. 2) and the cell growth factor 25 (see FIG. 2).

With such cell culture medium 8, the production cost of the useful substances may be reduced than before more reliably, since the cell growth factor 25 is an expensive medium component.

Alternatively, as the cell growth factor 25 (see FIG. 2) of the conjugate 21A (see FIG. 2) contained in the cell culture medium 8 (see FIG. 1) of this embodiment, employable is insulin or transferrins. Since insulin and transferrins are relatively expensive among the cell growth factors 25, so that the production cost of the useful substances may be reduced than before more reliably.

Moreover, the cell culture apparatus 1 (see FIG. 1) and the cell culture method, each using the aforementioned cell culture medium 8 (see FIG. 1), demonstrate not only operations and effects same as those of the cell culture medium 8 (see FIG. 1) described above, but also operations and effects below.

In the prior continuous culture described in Patent Literature 1, it might be possible to collect the cell culture medium 8 in an independent process, typically on the downstream side of a filter membrane (separation mechanism) that collects the cell, although such collection of cell culture medium 8 has not been mentioned. This however needs an additional separation mechanism for collecting the cell culture medium 8, in addition to the filter membrane (separation mechanism) that collects the cells.

In contrast, the cell culture apparatus 1 (see FIG. 1) and the cell culture method in this embodiment can separate and collects the cells and the conjugate 21As using a single separation mechanism 5 (see FIG. 1) in a single separation step.

As a consequence, this embodiment can simplify constituents of the cell culture apparatus 1 (see FIG. 1) and the cell culture method than before.

The embodiments of the present invention have been described. The present invention is, however, not limited to these embodiments, and may be embodied in various ways. Note that all constituents in other embodiments (modified examples) identical to those in the aforementioned embodiment will be given same reference symbols, and will not be detailed.

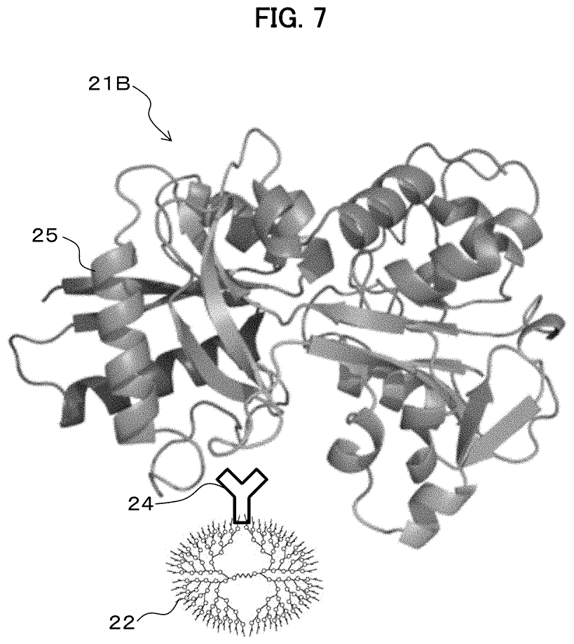

FIG. 5 is a schematic drawing of a conjugate 21B according to a modified example. FIG. 6 is an explanatory structural drawing illustrating a specific example of the conjugate 21B. FIG. 7 is a schematic drawing illustrating a state in the stimulus-applying step of the conjugate 21B illustrated in FIG. 5.

The conjugate 21A (see FIG. 2) in the aforementioned embodiment is composed of the stimuli-responsive polymer 22 (see FIG. 2) and the cell growth factor 25 (see FIG. 2).

In contrast, the conjugate 21B according to the modified example is composed of the stimuli-responsive polymer 22, and the binding factor 24 that binds to the cell growth factor 25 (see FIG. 2), as illustrated in FIG. 5.

The stimuli-responsive polymer 22 of the conjugate 21B may be composed in the same way as the stimuli-responsive polymer 22 (see FIG. 2) in the aforementioned embodiment.

The binding factor 24 of the conjugate 21B does not bind to the cell growth factor 25 in the aforementioned cell culture step, but binds to the cell growth factor 25 when the stimuli-responsive polymer 22 is stimulated in the aforementioned stimulus-applying step. Reference symbol 26 in FIG. 5 denotes a hydrophilic group, and may be composed in the same way as the hydrophilic group 26 of the conjugate 21A illustrated in FIG. 2.

The binding factor 24 is not specifically limited so long as it can specifically bind to the cell growth factor 25, and is exemplified by antibody, enzyme, protein, glycan, and nucleic acid that can specifically bind to the cell growth factor 25. Among them, antibody is preferable.

When the conjugate 21B according to the modified example is placed under a predetermined stimulus in the aforementioned stimulus-applying step, the cell growth factor 25 binds to the binding factor 24, and the stimuli-responsive polymer 22 concurrently changes as illustrated in FIG. 6. In this way, the conjugate 21B may be separable by the separation mechanism 5 (see FIG. 1). More specifically, the stimuli-responsive polymer 22 that is composed, for example, of temperature responsive polymer, pH responsive polymer, ionic strength responsive polymer, photoresponsive polymer, magnetic field responsive polymer, electric field responsive polymer, or dynamic stimulation responsive polymer can change structure, potential, or hydrophilicity/lipophilicity in response to the stimulus as described above, and turns the conjugate 21B into separable by the separation mechanism 5 (see FIG. 1).

FIG. 7 is an explanatory structural drawing illustrating a specific example of the conjugate 21B after being stimulated.

As illustrated in FIG. 7, the conjugate 21B is formed by dendritic polylysine as the stimuli-responsive polymer 22 and an antibody as the binding factor 24, which are chemically bound to each other. When temperature stimulation is applied to such conjugate 21B, the antibody as the binding factor 24 binds to a transferrin as the cell growth factor 25 through chemical bond.

With the cell culture medium 8 (see FIG. 1) containing such conjugate 21B (see FIG. 5), the expensive cell growth factor 25 (see FIG. 5) may be kept staying in the culture vessel 2 with the aid of the separation mechanism 5, when the cell culture medium 8 is drawn out together with the useful substance from the culture vessel 2 (see FIG. 1). This successfully reduces the feed volume of the expensive medium component to be supplemented to the culture vessel 2. Hence with the cell culture medium 8 of this embodiment, the production cost of the useful substances obtained by cell culture may be reduced than before.

### EXAMPLES

Examples of the present invention will be explained below.

### Example 1

First, a cell culture apparatus used to implement the cell culture method, described later in Example 2, will be explained.

FIG. 8 is an explanatory structural drawing illustrating a cell culture apparatus 1 used in Example 2.

As illustrated in FIG. 8, the cell culture apparatus 1 comprises the culture vessel 2 that stores the cell culture medium 8; the separation mechanism 5 that separates the later-described cultured cells and the conjugates from the cell culture medium 8 drawn out from the culture vessel 2; and the stimuli-applying mechanism 4 that applies temperature stimulus to the cell culture medium 8 drawn out from the culture vessel 2.

In this cell culture apparatus 1, the cell culture medium 8 (liquid medium) that contains the later-described conjugates (see Example 2 and Example 3) and other medium components necessary for the cell culture is stored in the culture vessel 2. Cells are cultured in the cell culture medium 8 at a predetermined culture temperature (20° C. or around in this Example), and the useful substances and the cell metabolites (waste products) are produced in the cell culture medium 8 as described above. In FIG. 8, reference symbol 9 denotes a stirrer. The stirrer 9 stirs the content of the culture vessel 2 into uniformity.

After an elapse of a predetermined number of days after the start of culture, or at a point in time when the concentration of the cultured cell in the culture vessel 2 reaches a predetermined level, the cell culture medium 8 in the culture vessel 2 is transferred to the separation mechanism 5. The cell culture medium 8 is transferred to the separation mechanism 5 mainly by elevating the inner pressure of the culture vessel 2 using an unillustrated pump or the like provided to the culture vessel 2. In FIG. 8, reference symbol 31 denotes a pressurizing mechanism typically composed of a diaphragm pump. The pressurizing mechanism 31 accelerates transfer of the cell culture medium 8 from the culture vessel 2 towards the separation mechanism 5, by setting the inner pressure of the separation mechanism 5 to a negative value.

The stimulus-applying mechanism 4 of this Example is composed of a heater. The stimuli-applying mechanism 4 heats the cell culture medium 8 on the way from the culture vessel 2 to the separation mechanism 5, to set the medium to a predetermined temperature (37° C. or around, in this Example). In this way, temperature stimulus is applied to the later-described conjugates, thereby the stimuli-responsive polymers of the conjugates lose the steric structures and elongates.

The separation mechanism 5 of this Example is composed of a hollow fiber filter module. The filtrate 23 after separated from the cultured cells and the later-described conjugate is collected in an unillustrated reservoir, and then subjected to the aforementioned purification step.

After a predetermined amount of cell culture medium 8 was subjected to the filtration step, or after the purification step was allowed to proceed for a predetermined duration in the separation mechanism 5, the cultured cell and the later-described conjugates collected by filtration are returned back to the culture vessel 2, together with a small amount of the cell culture medium 8 that remains in the separation mechanism 5. Operation of such returning is enabled when the pressurizing mechanism 31, which is composed of a diaphragm pump or the like, sets the inner pressure of the separation mechanism 5 to a positive value.

Next, the cell culture apparatus 1 supplements the supplementary medium 12, whose volume is comparable to that of the cell culture medium 8 having been drawn out from the culture vessel 2, to the culture vessel 2 at a point in time explained below.

In FIG. 8, a pipe which the supplementary medium 12 is fed from an unillustrated supplementary medium vessel towards the culture vessel 2, and reference symbol 32 denotes an on-off valve provided to the pipe. Reference symbol 33 denotes a sensor that detects liquid surface level in the culture vessel 2, and reference symbol 10 denotes a control device that controls open/close of the on-off valve 32 in a predetermined timing.

When the predetermined amount of the cell culture medium 8 drawn out by the separation mechanism 5 from the culture vessel 2 is subjected to the filtration step, the liquid surface level in the culture vessel 2 descends. Upon input of a signal from the sensor 33, the control device 10 determines that the cell culture medium 8 was drawn out from the culture vessel 2. Upon such determination, the control device 10 opens the on-off valve 32, and operates for example a pump provided to an unillustrated supplementary medium vessel, to transfer the supplementary medium 12 from the supplementary medium vessel to the culture vessel 2. The control device 10, when determines upon input of a signal from the sensor 33 that the culture vessel 2 was filled with a predetermined amount of the cell culture medium 8, stops the aforementioned pump, and closes the on-off valve 32.

Note that in the cell culture apparatus 1, the step of supplementing the supplementary medium 12 into the culture vessel 2 may take place concurrently with the filtration step by the separation mechanism 5 as described above.

Alternatively, the cell culture apparatus 1 may be designed to interrupt the separation step when the supplementation step is ongoing, and to restart the separation step after completion of the supplementation step.

Although not illustrated in FIG. 8, the control device 10 may be linked in a wired or wireless manner with the temperature regulating mechanism, a concentration measuring instrument and so forth in the cell culture apparatus 1 (see FIG. 1) of the aforementioned embodiment, so as to control various timings including timing of aeration by the aeration unit, timing of transfer of the cell culture medium 8, timing of return of the cell and the conjugate, and timing of filtration by the separation mechanism 5.

### Example 2

In this embodiment, the cell culture method below was implemented.

First, two types of conjugate of temperature responsive polymer as the stimuli-responsive polymer with the cell growth factor (medium component), namely a first conjugate and a second conjugate, were prepared.

### [First Conjugate]

First, ε-polylysine, which is a temperature responsive polymer, was prepared as the stimuli-responsive polymer 22 (see FIG. 2). The ε-polylysine was prepared by adding polylysine with a variety of molecular weights to an aqueous solution containing 1-ethyl-3-carbodiimide (WSC), N-hydroxysuccinimide (NHS), and valeric acid, and by removing low-molecular-weight unreacted compounds through a dialysis membrane. The thus prepared ε-polylysine was found to have a molecular weight of 50 kDa or smaller. To the ε-polylysine, hydrophilic groups composed of polyethylene glycol were bound as side chains.

Next, the ε-polylysine thus prepared as a temperature responsive polymer was added to an aqueous solution containing 1-ethyl-3-carbodiimide (WSC), N-hydroxysuccinimide (NHS) and insulin, to obtain a first conjugate with ε-polylysine and insulin which are chemically bound each other.

### [Second Conjugate]

First, ε-polylysine, which is a temperature responsive polymer, was prepared in the same way as the first conjugate was prepared, except that type of the dialysis membrane was changed to obtain ε-polylysine with a molecular weight of 100 kDa or larger.

Next, the ε-polylysine thus prepared as a temperature responsive polymer was added to an aqueous solution containing 1-ethyl-3-carbodiimide (WSC), N-hydroxysuccinimide (NHS), and anti-transferrin antibody as a binding factor, to obtain a second conjugate with the ε-polylysine and the anti-transferrin antibody which are chemically bound each other.

### [Cell Culture]

As a cell to be cultured, Chinese hamster ovary cell (CHO cell; CRL-9606) that produces tissue plasminogen activator (tPA), a kind of glycoprotein was obtained (from American Type Culture Collection (ATCC)). Note that the cell is acclimatized from adherent cultured cell into suspended cell.

As a cell culture medium, employed was Ham's F12 basal medium, further added with fatal bovine serum up to a final concentration of 10%, penicillin and streptomycin which are antibiotics, 10 μg/mL on an insulin basis of the first conjugate, 10 μg/mL on a transferrin basis of the second conjugate, and amino acid, vitamin and so forth.

Using this cell culture medium, the cell was cultured in the cell culture apparatus 1 of Example 1.

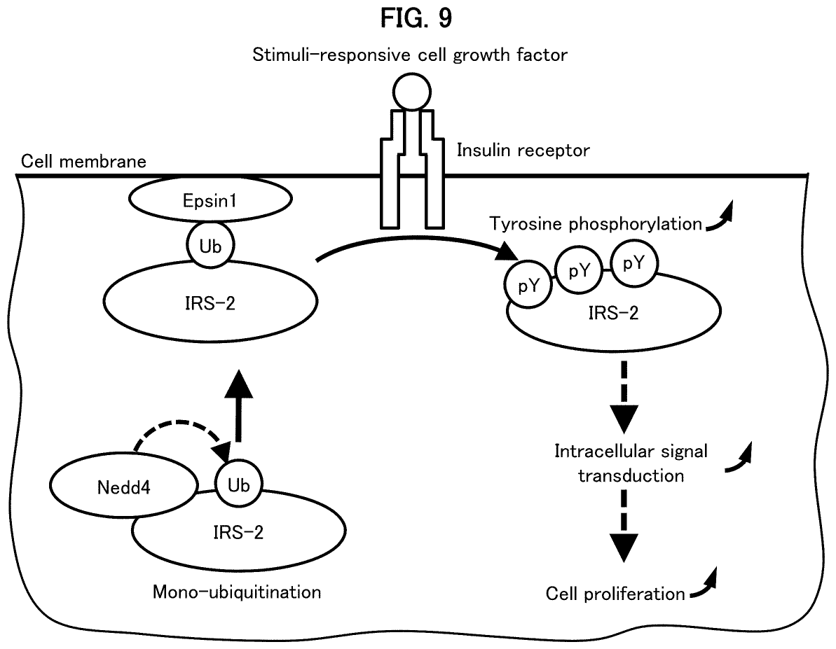

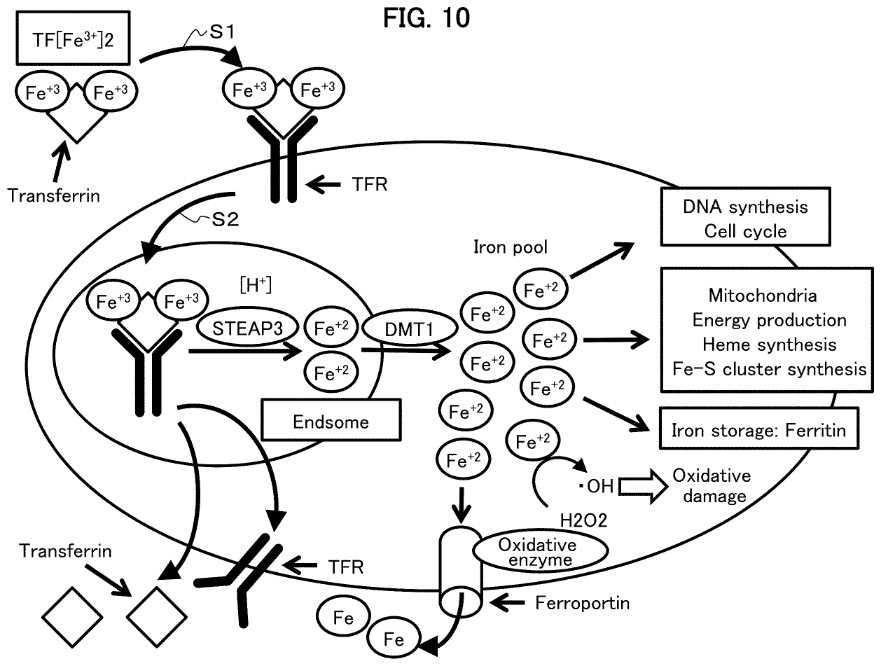

FIG. 9 is a chart illustration of intracellular mechanism of action of the first conjugate. FIG. 10 is a chart illustration of intracellulara mechanism of action of the second conjugate.

As illustrated in FIG. 9, an intracellular signal of insulin, which is important for control of metabolism or growth, is transduced through phosphorylation of an insulin receptor substrate (IRS) by a tyrosin kinase contained in an insulin receptor. Ubiquitin ligase Nedd4, as one protein that interacts with the IRS, monoubiquitinates the IRS, by which the IRS is moved closer to a receptor to enhance an insulin signal.

As illustrated in FIG. 10, when a transferrin having ferric ions bound binds to a transferrin receptor (TFR) that is situated in a cell membrane (see S1), this complex is incorporated into a cell by endocytosis (see S2). In an acidic environment in an endosome, the iron ions are released from the transferrin, so that ferric ions are reduced to ferrous ions. The transferrin freed from iron and the transferrin receptor are returned back to the cell membrane, and recycled.

Ferrous ions exit the endosome through a divalent metal transporter (DMT1) and move to the cytoplasm, and enter a labile iron pool that can be utilized for various intracellular functions. The iron ions are used for various intracellular purposes. Excessive iron ions are stored in ferritin which is an iron storage protein. The iron ions are excreted out of the cell, with the aid of ferroportin, an iron efflux pump.

Meanwhile, succeeding to the culturing step, the cell culture medium 8 (see FIG. 8) in the culture vessel 2 (see FIG. 8) is heated by the stimulus-applying mechanism 4 (see FIG. 8) from 20° C. up to 37° C., and pumped to the separation mechanism 5 (see FIG. 8).

Hence, the ε-polylysine, which is a temperature responsive polymer as the stimuli-responsive polymer 22 (see FIG. 2 or FIG. 5) in the first conjugate and the second conjugate, will have an increased molecular size, and will have electric charge turned into positive.

In the separation mechanism 5, the first conjugate and the second conjugate remained unfiltered, meanwhile tissue plasminogen activator as a useful substance, and, ammonia and lactic acid as the cell metabolites (waste products) were separated in the filtrate.

Insulin and transferrin, which are expensive medium components, were returned in the forms of first conjugate and second conjugate, back into the culture vessel 8 (see FIG. 8).

After one-month continuous operation of the cell culture apparatus 1 (see FIG. 8) of this Example, the tissue plasminogen activator as a useful substance was found to be collected with a high yield ratio, with 80% or more of the cells kept alive. In addition, the amounts of consumption of insulin and transferrin could largely be reduced.

### REFERENCE SIGNS LIST

1 cell culture apparatus
2 culture vessel
3 supplementary medium vessel
4 stimulus-applying mechanism
5 separation mechanism
6 reservoir
7 purification mechanism
8 cell culture medium
9 stirrer
10 control device
11 aeration unit
12 supplementary medium
21A conjugate
21B conjugate
22 stimuli-responsive polymer
23 filtrate
24 binding factor
25 cell growth factor
26 hydrophilic group
31 pressurizing mechanism

What is claimed is:

1. A cell culture medium comprising at least one of medium components that is a conjugate with a stimuli-responsive polymer.

2. The cell culture medium as claimed in claim 1, wherein the stimuli-responsive polymer changes in response to at least one stimulus of temperature, light and pH.

3. The cell culture medium as claimed in claim 2, wherein the stimuli-responsive polymer, at least, elongates its molecular chain or changes its polarization charge in response to the stimulus.

4. The cell culture medium as claimed in claim 1, wherein the stimuli-responsive polymer is at least one of polylysine, polyglutamine and polyarginine.

5. The cell culture medium as claimed in claim 1, wherein the conjugate is composed of the stimuli-responsive polymer and a cell growth factor.

6. The cell culture medium as claimed in claim 5, wherein the cell growth factor is at least one growth factor selected from the group consisting of epidermal growth factor (EGF), insulin-like growth factor (IGF), transforming growth factors (TGF), nerve growth factor (NGF), brain-derived neurotrophic factor (BDNF), vesicular endothelial growth factor (VEGF), Glanulocitye-Colony Stimulating Factor (G-CSF), Granulocyte-Macrophage-Colony Stimulating Factor (GM-CSF), Platelet-Derived Growth Factor (PDGF), erythropoietin (EPO), thrombopoietin (TPO), basic fibroblast growth factor (bFGF or FGF2), hepatocyte growth factor (HGF), transforming growth factor (TGF), bone morphogenic proteins (BMP), brain-derived neurotrophic factor (BDNF, NGF or NT3), fibroblast growth factors (FGF), serum, Bovine Serum Albumin (BSA), cholesterols, insulin and transferrins.

7. The cell culture medium as claimed in claim 1, wherein the conjugate is composed of the stimuli-responsive polymer and a binding factor that binds to the cell growth factor.

8. The cell culture medium as claimed in claim 7, wherein the binding factor is at least one of an antibody of the cell growth factor, an enzyme, a protein, a glycan and a nucleic acid.

9. A cell culture apparatus comprising:
a culture vessel where a cell culture medium comprising at least one of the medium components composed of a conjugate with a stimuli-responsive polymer is stored and cells are cultured in the medium;
a stimulus-applying mechanism which applies a predetermined stimulus to the conjugate so as to induce a predetermined change of the stimulus-responsive polymer in response to the stimulus; and
a separation mechanism which separates at least a part of the medium components except for the conjugate from the cell culture medium, while leaving the conjugate in the cell culture medium, on the basis of a property change of the stimuli-responsive polymer.

10. A cell culture method comprising:
a cell culture step in which cells are cultured in a cell culture medium comprising at least one of the medium components composed of a conjugate with a stimuli-responsive polymer;
a stimulus-applying step in which a predetermined stimulus is applied to the conjugate so as to induce a predetermined change of the stimuli-responsive polymer; and,
a separating step in which at least a part of the medium components except for the conjugate is separated from the cell culture medium, while leaving the conjugate in the cell culture medium, on the basis of a property change of the stimuli-responsive polymer.
